(19) United States
(12) Patent Application Publication
(10) Pub. No.: US 2025/0082647 A1
(43) Pub. Date: Mar. 13, 2025

(54) SPECIFIC DOSAGE OF PHENOTHIAZINE COMPOUNDS FOR USE IN TREATMENT OR PREVENTION OF ALZHEIMER'S DEMENTIA

(71) Applicant: IMMUNGENETICS AG, Hamburg (DE)

(72) Inventor: Markus KROHN, Heidelberg (DE)

(21) Appl. No.: 18/726,500

(22) PCT Filed: Jan. 3, 2023
(86) PCT No.: PCT/EP2023/050065

§ 371 Date: Jul. 3, 2024

(30) Foreign Application Priority Data
Jan. 4, 2022 (EP) 22150214.9

(51) Int. Cl. A61K 31/5415

(52) U.S. Cl. CPC A61K 31/5415

(57) ABSTRACT

The present invention relates in a first aspect to a compound of formula (I) for use in the treatment and/or in prevention of Alzheimer's dementia, wherein the compound of formula (I) is administered in a dosage<20 mg. In a second aspect, the invention relates to a pharmaceutical composition comprising the compound according to the first aspect.

The present invention relates in a first aspect to a compound of formula (I) for use in the treatment and/or in prevention of Alzheimer's dementia, wherein the compound of formula (I) is administered in a dosage≤20 mg. In a second aspect, the invention relates to a pharmaceutical composition comprising the compound according to the first aspect.

EP 2 614 060 B1 (WO 2012/031941 A2) discloses thiethylperazine (IUPAC: 2-(ethylsulfanyl)-10-[3-(4-methylpiperazin-1-yl)propyl]-10H-phenothiazine, CAS: 1420-55-9) and derivatives thereof for treatment of a beta-amyloidopathy or an alpha-synucleinopathy accompanied by a cerebral protein deposit and a reduced activity of the cerebral ABCC1-transporter. As also described in Krohn et al., thiethylperazine activates the transport protein ABCC1 that clears beta-amyloid from brains of mice (Krohn, M., C. Lange, J. Hofrichter, K. Scheffler, J. Stenzel, J. Steffen, T. Schumacher, T. Bruning, A. S. Plath, F. Alfen, A. Schmidt, F. Winter, K. Rateitschak, A. Wree, J. Gsponer, L. C. Walker and J. Pahnke (2011). “Cerebral amyloid-beta proteostasis is regulated by the membrane transport protein ABCC1 in mice.” J Clin Invest 2011 121(10): 3924-3931). Thus, thiethylperazine has been proven effective in murine models of Alzheimer's disease (AD) to export amyloid beta (Abeta) from and through the endothelial cells of the blood-brain barrier to the periphery. It has also been shown that pharmaceutical activation of ABCC1 can reduce amyloid plaque deposition in the brain. Thiethylperazine had been used in this study for mice in a concentration of 15 mg/kg body weight per day, which would, considering an allometric scaling factor of 7 between mouse and human, result in a human equivalent dose of 105 mg thiethylperazine per day.

Jepsen, De Both et al. showed that ABCC1 is not only capable of exporting Abeta from the cytoplasm of human cells, but also that its overexpression significantly reduces Abeta production and increases the ratio of alpha- versus beta-secretase mediated cleavage of the amyloid precursor protein (APP), likely via indirect modulation of alpha-, beta- and gamma-secretase activity. When incubated with fluorescent Abeta-42, 79.7% of wild-type human BE(2)-m17 neuroblastoma cells and 68.4% of ABCC1 overexpressing BE(2)-m17 cells become fluorescent. Treatment with thiethylperazine lead to a 29.6% decrease of wild-type cells that efficiently took up Abeta-42. Simultaneous overexpression of ABCC1 enhanced this effect to 44.4%. Hence, ABCC1 effectively protects cells from Abeta entry and thiethylperazine amplifies this effect (Jepsen, W. M., M. De Both, A. L. Siniard, K. Ramsey, I. S. Piras, M. Naymik, A. Henderson and M. J. Huentelman (2020). “Adenosine triphosphate Binding Cassette subfamily C member 1 (ABCC1) overexpression reduces APP processing and increases alpha- versus beta-secretase activity, in vitro.” Biol Open: bio.054627). Furthermore, ABCC1 overexpressing BE(2)-m17 cells produce less Abeta-42 and Abeta-40 through shifting APP processing towards the non-amyloidogenic pathway (Jepsen, De Both et al.).

Regarding beta-amyloidopathies such as Alzheimer's disease (and Parkinson's disease), thiethylperazine is considered as being potentially able to delay the progression of Alzheimer's dementia and Parkinson's disease by several years. The drug is in Phase II clinical trials under EudraCT Number: 2014-000870-20. In view of what is considered to be needed with respect to efficiency, the clinical trials were made with 26 mg of thiethylperazine per person and day and with 52 mg of thiethylperazine per person and day respectively. However, in view of the increase of Alzheimer's disease and the increasing life expectancy as well as an increasing proportion of older people in the total population, there is still a need for improved formulations for the treatment of Alzheimer's disease.

The objective technical problem underlying the present invention was thus the provision of an improved formulation for (use in) treatment of Alzheimer's disease and/or in the prevention of Alzheimer's disease.

### 1^{st} Aspect—Compound of Formula (I) for Use in the Treatment and/or in the Prevention of Alzheimer's Dementia

In a first aspect, the present invention relates to a compound of formula (I) for use in the treatment and/or in the prevention of Alzheimer's dementia,

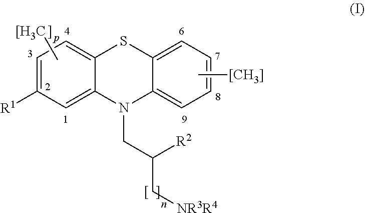

wherein:
R¹ is selected from the group consisting of hydrogen atom, halogen atom, methoxy group, perhalogenated alkyl group and —X-C1 to C2 alkyl group, wherein X is either a sulphur atom or an oxygen atom;
R² is a hydrogen atom or a methyl group;
R³, R⁴ are independently of each other either a hydrogen atom or C1 to C4 alkyl group or form together a six-membered alkyl ring which comprises at least one further nitrogen atom, wherein the at least one further nitrogen is substituted with a C1 to C3 alkyl-R⁵ group, wherein R⁵ is a hydrogen atom or a hydroxyl group;
n is zero or 1;
m is zero or an integer selected from the range of from 1 to 4;
p is zero or an integer selected from the range of from 1 to 3;
wherein the compound of formula (I) is administered in a dosage≤20 mg per day of administration.

Preferably, the compound of formula (I) for use in the treatment and/or in the prevention of Alzheimer's dementia, is administered in a dosage in the range of from 2 to 20 mg per day of administration. “Per day of administration” means that the dosage of ≤20 mg is administration on in the range of from 1 to 7 day(s) per week, preferably either every day (on 7 days per week, i.e. daily) or every second day or every third day, more preferably the dosage of ≤20 mg is given every day or every second day, more preferably the dosage of ≤20 mg is given every day.

In some embodiments, the [CH₃]ₘ-group(s) is/are preferably present at a position selected from 6, 7, 8 and 9; the [CH₃]ₚ-group(s) is/are present at a position selected from 1, 3 and 4 of the phenothiazine structure. If R² is a methyl group, then the carbon atom carrying R² has preferably R configuration.

Normally, the skilled person will try to reduce the dosage of a drug as far as possible but it is expected by the skilled person that especially the efficacy of a treatment with an active agent (active pharmaceutical ingredient, API) decreases with decreasing amounts of active agent administered. Hence, the skilled person expects to trade a lower dosage (cheaper and safer treatment) of the drug for a lower therapeutic effect and a potentially smaller amount of responsive patients. It is a commonly accepted principle that the response in a patient is related to the dose such that increasing the dose produces a greater response until a maximum effect (Eₘₐₓ) is reached (see, for example, R. Lieberman et al. 1993). However, in the context of the present invention, it was surprisingly found that for a compound of formula (I) or pharmaceutically acceptable salt thereof, especially for thiethylperazine and more especially for maleate salt(s) of thiethylperazine, the efficacy, as well as the response to a treatment in humans are improving towards lower, not higher doses. Thiethylperazine is 2-(ethylsulfanyl)-10-[3-(4-methylpiperazin-1-yl)propyl]-10H-phenothiazine (CAS: 1420-55-9), thiethylperazine maleate (CAS: 1179-69-7, C₃₀H₃₇N₃O₈S₂) is the salt of (positively charged) thiethylperazine with two (negatively charged) molecules of maleate. Not only were surprisingly more subjects i.e. in the range of 30 to 117% more subjects, found to be responsive to a lower dose of thiethylperazine in the range of from 2-20 mg, but also the average increase in plasma Abeta1-40 was significantly higher, especially ≥20% higher compared to subjects, which had been treated with a higher amount of thiethylperazine. Furthermore, also the mean plasma Abeta1-40 concentration difference between responders and non-responders was substantially increased by ≥10 pg/ml in subjects, which had been treated with a lower dose of thiethylperazine in the range of from 2-20 mg compared to subjects, which had been treated with a higher amount of thiethylperazine.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may all refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The term “pharmaceutically acceptable”, as used herein, relates to a salt, which is pharmaceutically acceptable in the sense of being not deleterious to the recipient thereof and, preferably, being compatible with optional other ingredients of a formulation thereof. Preferably, a pharmaceutically acceptable salt is a salt of the compound of formula (I) causing at most moderate adverse drug reactions, preferably causing at most mild adverse drug reactions. As used herein, the term “mild” adverse reactions relates to adverse reactions not requiring medical intervention, such as skin rashes, headaches, digestive disturbances, fatigue, and the like; “moderate” adverse reactions are adverse reactions requiring medical intervention, but not being potentially life-threatening.

The term “alkyl”, as used herein, relates to a straight or branched chain, saturated hydrocarbon group, linked to the main chain by a covalent bond to at least one of its at least one carbon atoms. Preferred alkyl groups are straight-chain alkyls or branched chain alkyl groups. Accordingly, alkyl groups include primary alkyl groups, secondary alkyl groups, and tertiary alkyl groups.

The terms “treating” and “treatment” refer to an amelioration of the diseases or disorders referred to herein or the symptoms accompanied therewith to a significant extent. Said treating as used herein also includes an entire restoration of health with respect to the diseases or disorders referred to herein. It is to be understood that treating, as the term is used herein, may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well-known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 10%, at least 20% at least 50% at least 60%, at least 70%, at least 80%, or at least 90% of the subjects of a given cohort or population. Preferably, treating Alzheimer's disease is improving one or more biomarkers of neurodegeneration, which are known to the skilled person, like for example, Abeta-40, Abeta-42, or Abeta42/40 ratio, phosphorylated variants of the Tau-protein (e.g. p-Tau181, p-Tau217 etc.), Glial fibrillary acidic protein (GFAP), Neurofilament light chain (NfL) etc. in bodily fluids (blood, CSF), and/or improving Abeta and/or Tau brain burden determined using positron emission tomography (PET) methods, and/or slowing or stopping the decline of or improving of cognitive abilities as determined by one or more tests, which are known to the skilled person, like for example, ADAS-Cog, FCSRT, ADCS-iADL, QOL-AD, CDR, MMSE etc. in a subject. As will be understood by the skilled person, the effectiveness of treatment of Alzheimer's disease is dependent on a variety of factors including, e.g. stage and severity of the disease. “Improving one or more biomarkers” is to be understood in that the value of the respective biomarker after or during treatment of a subject deviates less from a reference value of a healthy subject than before treatment. “Slowing or stopping the decline of cognitive abilities or improving of cognitive abilities” relates to results from a respective test, which after or during treatment deviate less from a reference value of a healthy subject or a known reference value than before treatment.

The terms “preventing” and “prevention” refer to retaining health with respect to Alzheimer's disease for a certain period of time in a subject. It will be understood that the said period of time may be dependent on the amount of the drug compound which has been administered and individual factors of the subject discussed elsewhere in this specification. It is to be understood that prevention may not be effective in all subjects treated with the compound according to the present invention. However, the term requires that, preferably, a statistically significant portion of subjects of a cohort or population are effectively prevented from suffering from a disease or disorder referred to herein or its accompanying symptoms. Preferably, a cohort or population of subjects is envisaged in this context which normally, i.e. without preventive measures according to the present invention, would develop a disease or disorder as referred to herein. Whether a portion is statistically significant can be determined without further ado by the person skilled in the art using various well-known statistic evaluation tools discussed elsewhere in this specification.

The term “subject”, as used herein, relates to a multicellular animal, preferably to a vertebrate, more preferably to a mammal, more preferably a human.

In some embodiments of the use in the treatment and/or in the prevention of Alzheimer's dementia, R¹ of the compound of formula (I) is selected from the group consisting of hydrogen atom, chlorine atom, thiomethyl group, thioethyl group, methoxy group, ethoxy group and trifluoromethyl group, preferably from the group consisting of thiomethyl group, thioethyl group, methoxy group, ethoxy group and trifluoromethyl group.

In some embodiments of the use in the treatment and/or in the prevention of Alzheimer's dementia,
R¹ is selected from the group consisting of hydrogen atom, chlorine atom, methoxy group, trifluoromethyl group and thioethyl group;
R² is a hydrogen atom or a methyl group;
R³ and R⁴ are independently of each other a methyl or an ethyl group or form together a six-membered alkyl ring which comprises at least one further nitrogen atom, wherein the at least one further nitrogen is substituted with a C1 to C3 alkyl-R⁵ group, wherein R⁵ is a hydrogen atom or a hydroxyl group;
n is zero or 1;
m, p are each zero.

In some embodiments of the use in the treatment and/or in the prevention of Alzheimer's dementia, the compound of formula (I) is selected from the group consisting of levomepromazine, trifluoperazine, thiethylperazine, perphenazine, chlorpromazine, thioridazine, diethazine, and mixtures of two or more thereof. Each of these compounds is known to activate the ABCC1 transporter that clears beta-amyloid from the brain.

In some embodiments of the use in the treatment and/or in the prevention of Alzheimer's dementia, R³ and R⁴ form a six-membered ring comprising one further nitrogen atom, wherein the further nitrogen atom has a methyl group or a —CH₂—CH₂—OH group.

In some embodiments of the use in the treatment and/or in the prevention of Alzheimer's dementia, the compound has formula (Ia) thereof:

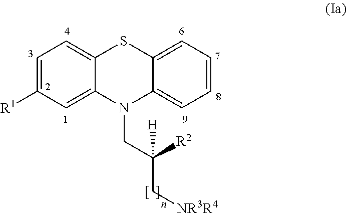

wherein
R¹ is selected from the group consisting of methoxy group, trifluoromethyl group and thioethyl group;
R² is a hydrogen atom or a methyl group (preferably having R configuration);
R³, R⁴ are both a methyl group or form together a six-membered alkyl ring, which comprises one further nitrogen atom, wherein the one further nitrogen is substituted with a methyl group,
n is zero or 1.

In some embodiments of the use in treatment and/or in the prevention of Alzheimer's dementia, R³, R⁴ of the compound of formula (Ia) are both a methyl group or form together a piperazine ring wherein the further nitrogen atom is substituted with a methyl group (4-methylpiperazin-1-yl group).

The compound of formula (I) or formula (Ia) is used as (uncharged) compound or is used in the form of a pharmaceutically acceptable salt thereof. A pharmaceutically acceptable salt of a compound of formula (I) or of a compound of formula (Ia) is administered so that the part of the cation corresponding to a compound of formula (I) or of a compound of formula (Ia) amounts to ≤20 mg, preferably in the range of from 2 to 20 mg, preferably per day of administration.

Preferably, a pharmaceutically acceptable salt comprises one or more anion(s) selected from the group consisting of sulfate, pyrosulfate, bisulfate, sulfite, bisulfite, phosphate, monohydrogen phosphate, dihydrogen phosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, malate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenyl acetate, phenyl propionate, phenyl butyrate, citrate, lactate, β-hydroxybutyrate, glycolate, tartrate, methanesulfonate, propanesulfonate, naphthalene-1-sulfonate, naphthalene-2-sulfonate, almondate, adipate, alginate, mucate, aspartate, benzenesulfonate, butyrate, camphorate, camphorsulfonate, cyclopentane propionate, digluconate, dodecyl sulfate, ethane sulfonate, glucoheptanoate, glycerophosphate, hemisulfate, hexanoate, 2-hydroxy-ethane sulfonate, 2-napthalene sulfonate, nicotinate, nitrate, palmitate, pectinate, persulfate, picrate, pivalate, salicylate, thiocyanate, tosylate, arginate, and undecanoate. More preferably, a pharmaceutically acceptable salt comprises one or more anion(s) selected from the group consisting of chloride, sulfate, mesylate, besylate, tosylate, fumarate, malonate, malate, maleate, succinate, lactate, glycolate, citrate, aspartate and mandelate, preferably malate and/or maleate, more preferably maleate.

In some embodiments, the compound of formula (I) or formula (Ia) is selected from the group consisting of levomepromazine, trifluoperazine and thiethylperazine and pharmaceutically acceptable salts of these compounds and is preferably levomepromazine or thiethylperazine or a pharmaceutically acceptable salt of levomepromazine or a pharmaceutically acceptable salt of thiethylperazine, and is more preferably thiethylperazine or a pharmaceutically acceptable salt of thiethylperazine.

Preferably, the compound of formula (I) or formula (Ia) is used alone or as a mixture of two or more compounds of the formula (I) or formula (Ia) or as a pharmaceutically acceptable salt of a compound of the formula (I) or formula (Ia) or as a mixture of two or more pharmaceutically acceptable salts of compounds of the formula (I) or formula (Ia). Likewise, mixtures of one or more compound(s) of formula (I) or formula (Ia) with one or more pharmaceutically acceptable salt(s) of compound(s) of the formula (I) or formula (Ia) can be used. In some preferred embodiments, the one or more compound(s) of formula (I) or formula (Ia) are only used in the form of their pharmaceutically acceptable salt(s), more preferably the one or more compound(s) of formula (I) or formula (Ia) are used in the form of their maleate and/or malate salts, more preferably the one or more compound(s) of formula (I) or formula (Ia) are used in the form of their maleate salts. The salt forms offer the advantage that they have improved solubility in aqueous systems.

As indicated above, the compound of formula (I) is used in the use in treatment and/or in the prevention of Alzheimer's dementia in a dosage≤20 mg, preferably in the range of from 2 to 20 mg, preferably per day of administration. In some embodiments of the use in treatment and/or in the prevention of Alzheimer's dementia, the compound of formula (I) or formula (Ia) is administered in a dosage in the range of from 5 to 15 mg, preferably in the range of from 6 to 13.5 mg, per day of administration. More preferably, the compound of formula (I) or formula (Ia) is administered in a dosage in the range of from 6 to 7 mg per day of administration or in the range of from 12.5 to 13.5 mg, per day of administration.

In the context of the present invention, if salts of the compound of formula (I) or formula (Ia) are used, the weight amounts indicated above are related to the compound of formula (I) or formula (Ia), i.e. the amount of the respective salt, which has to be used is, due to the higher molecular weight of the salt, higher. However, as indicated above, a pharmaceutically acceptable salt of a compound of formula (I) or of a compound of formula (Ia) is used so that the part of the cation corresponding to a compound of formula (I) or of a compound of formula (Ia) amounts to ≤20 mg, preferably in the range of from 2 to 20 mg, preferably per day of administration. For example, if thiethylperazine maleate is used, a range of from 2 to 20 mg for thiethylperazine corresponds to a range of from 3.16 to 31.62 mg of thiethylperazine maleate. If thiethylperazine malate (CAS: 52239-63-1, C₃₀H₄₁N₃O₁₀S₂) is used, which is the salt of (positively charged) thiethylperazine with two (negatively charged) molecules of malate, a range of from 2 to 20 mg for thiethylperazine corresponds to a range of from 3.34 to 33.42 mg of thiethylperazine maleate. The same applies to the preferred ranges and values. Thus, in some embodiments, the compound of formula (I) or formula (Ia) is used as pharmaceutically acceptable salt of formula (I) or formula (Ia), preferably as thiethylperazine maleate, preferably in a dosage≤31.62 mg, more preferably in the range of from 3.16 to 31.62 mg, more preferably in the range of from 7.9 to 23.72 mg, more preferably in the range of from 9.48 to 21.34 mg, of thiethylperazine maleate per day of administration. More preferably, thiethylperazine maleate is administered in a dosage in the range of from 9.47 to 11.07 mg thiethylperazine maleate per day of administration or in the range of from 19.77 to 21.34 mg thiethylperazine maleate per day of administration.

In some embodiments of the use in treatment and/or in the prevention of Alzheimer's dementia, the compound of formula (I) or formula (Ia) or pharmaceutically acceptable salt of formula (I) or formula (Ia) is administered as a single dose once per day of administration or twice per day of administration.

In some embodiments of the use in treatment and/or in the prevention of Alzheimer's dementia, the compound of formula (I) or formula (Ia) or pharmaceutically acceptable salt of formula (I) or formula (Ia) is administered orally or rectally.

### 2^{nd} Aspect—Pharmaceutical Composition

In a second aspect, the invention relates to a pharmaceutical composition comprising the compound of formula (I) or formula (Ia) as defined above in the section related to the first aspect of ≤20 mg, preferably in the range of from 2 to 20 mg, more preferably in the range of from 5 to 15 mg, more preferably in the range of from 6 to 13.5 mg. All definitions given and all preferred embodiments as described in the section related to the first aspect also apply for the pharmaceutical composition according to the second aspect.

The pharmaceutical composition may also, aside from the active compound, include at least one pharmaceutically acceptable excipient. Pharmaceutically acceptable excipients include, but are not limited to, carriers, adjuvants, stabilizers and/or other compounds deemed appropriate by the skilled person, e.g. for galenic purposes. As referred to herein, the compound of formula (I), (Ia) or the respective pharmaceutically acceptable salt as specified herein above in the section related to the first aspect is the “active compound” of the composition, although “further active compounds”, which are referred to under this term, may be present. Preferably, the further active compound is also a pharmaceutically active compound. Specific pharmaceutical compositions are prepared in a manner well known in the pharmaceutical art and comprise at least one active compound referred to herein above, preferably in admixture or otherwise associated with at least one pharmaceutically acceptable carrier or diluent. For making those specific pharmaceutical compositions, the active compound(s) will usually be mixed with a carrier or the diluent. The resulting formulations are to be adapted to the mode of administration, i.e. in the forms of tablets, capsules, suppositories, solutions, suspensions or the like as outlined for specific preferred embodiments in more detail below.

In one embodiment, the pharmaceutical composition is a unit dosage preparation comprising a compound of formula (I) or formula (Ia) or pharmaceutically acceptable salt of formula (I) or formula (Ia) as defined above in the section related to the first aspect and a pharmaceutically acceptable excipient. “Unit dosage preparation,” “unit dose” or “unit dose form” means a single dose of compound of formula (I) formula (Ia) or a pharmaceutically acceptable salt of compound of formula (I) formula (Ia), which is capable of being administered to a subject, and which can be readily handled and packaged, remaining as a physically and chemically stable unit dose.

The unit dosage preparation contains ≤20 mg of the compound of formula (I) or formula (Ia), preferably from 2 to 20 mg of the compound of formula (I) or formula (Ia), more preferably from 5 to 15 mg of the compound of formula (I) or formula (Ia), more preferably from 6 to 13.5 mg of the compound of formula (I) or formula (Ia). More preferably, the unit dosage preparation contains in the range of from 6 to 7 mg or in the range of from 12.5 to 13.5 mg of the compound of formula (I) or formula (Ia). In some embodiments, wherein the compound of formula (I) or formula (Ia) is administered as a single dose once per day of administration, the unit dosage preparation is designed to contain the complete amount of the compound of formula (I) or formula (Ia) in one unit dosage preparation. In some embodiments, wherein the compound of formula (I) or formula (Ia) is administered twice per day of administration, the unit dosage preparation is designed to contain half of the complete amount of the compound of formula (I) or formula (Ia) per unit dosage preparation.

In some embodiments, when a pharmaceutically acceptable salt of a compound of formula (I) or formula (Ia) used, the part of the cation corresponding to a compound of formula (I) or of a compound of formula (Ia) amounts to ≤20 mg, preferably in the range of from 2 to 20 mg in the unit dosage preparation. In some embodiments, when thiethylperazine maleate is used, the unit dosage preparation comprises ≤31.62 mg, more preferably in the range of from 3.16 to 31.62 mg, more preferably in the range of from 7.9 to 23.72 mg, more preferably in the range of from 9.48 to 21.34 mg, of thiethylperazine maleate. More preferably, the unit dosage preparation comprises thiethylperazine maleate in an amount in the range of from 9.47 to 11.07 mg thiethylperazine maleate or in the range of from 19.77 to 21.34 mg thiethylperazine maleate. In some embodiments, wherein the pharmaceutically acceptable salt of a compound of formula (I) or formula (Ia) is administered as a single dose once per day of administration, the unit dosage preparation is designed to contain the complete amount of the pharmaceutically acceptable salt of a compound of formula (I) or formula (Ia) in one unit dosage preparation. In some embodiments, wherein the pharmaceutically acceptable salt of a compound of formula (I) or formula (Ia) is administered twice per day of administration, the unit dosage preparation is designed to contain half of the complete amount of the pharmaceutically acceptable salt of a compound of formula (I) or formula (Ia) per unit dosage preparation.

In some embodiments, the pharmaceutical composition comprising the compound of formula (I) or formula (Ia) or pharmaceutically acceptable salt of formula (I) or formula (Ia) is in the form of a tablet or a suppository. A tablet preferably further comprises one or more auxiliary agents, preferably selected from the group consisting of gum arabic, gelatin, talc, stearic acid, corn starch, lactose, sucrose, β-carotene and peanut oil. In some embodiments, a tablet comprises one or more coatings on its outer surface, wherein the coating(s) materials are selected from sugars, polymeric materials and mixtures of sugar and polymeric material. A sugar-coating comprises one or more sugar(s), preferably selected from the group of sucrose, glucose, lactose, maltitol, mannitol, isomalt, sorbitol, xylitol and starch. A polymeric coating preferably comprises one or more polymer(s), preferably selected from the group consisting of cellulose ethers, vinyl polymers, glycols, and acrylic acid (co)polymers, wherein suitable polymers are known to the skilled person. A coating optionally comprises one or more further components, preferably selected from filler, colorant, antiadhesive, lubricant, glidant, flavoring agent, suspension stabilizer, smoothing agent, and polishing agent, wherein suitable substances therefore are known to the skilled person.

A suppository preferably further comprises one or more auxiliary agents, preferably selected from the group consisting of lactose, preferably lactose monohydrate and solid semisynthetic glycerides.

In some embodiments, the pharmaceutical composition comprising the compound of formula (I) or formula (Ia) or pharmaceutically acceptable salt of formula (I) or formula (Ia) is in the form of a solution, preferably a solution suitable for injection.

A solution suitable for injection preferably further comprises water and one or more components selected from the group of sodium metabisulfite, ascorbic acid, and sorbitol. Further, a solution suitable for injection has preferably a pH value in <5, preferably <4, more preferably <3, wherein the pH value is preferably determined by a pH-sensitive glass electrode according to DIN 19263:2007-05.

In some embodiments, the pharmaceutical composition comprising the compound of formula (I) or formula (Ia) or pharmaceutically acceptable salt of formula (I) or formula (Ia) is in the form of an aerosol, preferably an aerosol suitable for spray application.

### Method of Treatment

The present invention also relates to a method for treating and/or preventing Alzheimer's dementia comprising
(a) administering a compound of formula (I)

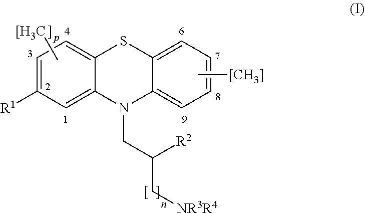

wherein:
R¹ is selected from the group consisting of hydrogen atom, halogen atom, methoxy group, perhalogenated alkyl group and —X-C1 to C2 alkyl group, wherein X is either a sulphur atom or an oxygen atom;
R² is a hydrogen atom or a methyl group;
R³, R⁴ are independently of each other either a hydrogen atom or C1 to C4 alkyl group or form together a six membered alkyl ring which comprises at least one further nitrogen atom, wherein the at least one further nitrogen is substituted with a C1 to C3 alkyl-R⁵ group, wherein R⁵ is a hydrogen atom or a hydroxyl group;
n is zero or 1;
m is zero or an integer selected from the range of from 1 to 4;
p is zero or an integer selected from the range of from 1 to 3;
wherein the compound of formula (I) is administered in a dosage≤20 mg, preferably in the range of from 2 to 20 mg, preferably per day of administration;
(b) thereby treating and/or preventing Alzheimer's dementia.

In some embodiments the invention relates to a method for treating and/or preventing Alzheimer's dementia comprising administering a pharmaceutical composition such as described above in the section related to the second aspect. All details described in the sections related to the first and the second aspect also apply here for the method of treatment and/or prevention.

The method for treating and/or prevention, preferably, is an in vivo method. Moreover, it may comprise steps in addition to those explicitly mentioned above. For example, further steps may relate, e.g., to diagnosing Alzheimer's dementia and/or identifying the subject as profiting from treatment with a compound of formula (I), preferably according to the method specified above, before step (a); thus, preferably, the method comprises the further steps of (i) determining or having determined Alzheimer's dementia in a subject; and (ii) identifying or having identified a subject profiting from treatment with compound of formula (I). Also, a further step may comprise co-treating the subject as specified herein above in step (b). Moreover, one or more of said steps may be performed by automated equipment.

The present invention is further illustrated by the following embodiments and combinations of embodiments as indicated by the respective dependencies and back-references. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”.

According to an embodiment (1), the present invention relates to a compound of formula (I) for use in the treatment and/or in the prevention of Alzheimer's dementia,

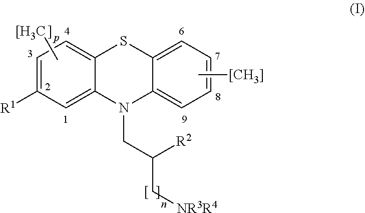

wherein:
R¹ is selected from the group consisting of hydrogen atom, halogen atom, methoxy group, perhalogenated alkyl group and —X-C1 to C2 alkyl group, wherein X is either a sulphur atom or an oxygen atom;
R² is a hydrogen atom or a methyl group;
R³, R⁴ are independently of each other either a hydrogen atom or C1 to C4 alkyl group or form together a six-membered alkyl ring which comprises at least one further nitrogen atom, wherein the at least one further nitrogen is substituted with a C1 to C3 alkyl-R⁵ group, wherein R⁵ is a hydrogen atom or a hydroxyl group;
n is zero or 1;
m is zero or an integer selected from the range of from 1 to 4;
p is zero or an integer selected from the range of from 1 to 3;
wherein the compound of formula (I) is administered in a dosage≤20 mg per day of administration.

A preferred embodiment (2) concretizing embodiment (1) relates to said compound of formula (I) for use according to embodiment (1), wherein the compound of formula (I) is administered in a dosage in the range of from 2 to 20 mg per day of administration.

A preferred embodiment (3) concretizing embodiment (1) or (2) relates to said compound of formula (I) for use according to embodiment (1) or (2), wherein R¹ is selected from the group consisting of hydrogen atom, chlorine atom, thiomethyl group, thioethyl group, methoxy group, ethoxy group and trifluoromethyl group, preferably from the group consisting of thiomethyl group, thioethyl group, methoxy group, ethoxy group and trifluoromethyl group.

A preferred embodiment (4) concretizing any one of embodiments (1) to (3) relates to said compound of formula (I) for use according to any one of embodiments (1) to (3), wherein
R¹ is selected from the group consisting of hydrogen atom, chlorine atom, methoxy group, trifluoromethyl group and thioethyl group;
R² is a hydrogen atom or a methyl group
R³ and R⁴ are independently of each other a methyl or an ethyl group or form together a six-membered alkyl ring which comprises at least one further nitrogen atom, wherein the at least one further nitrogen is substituted with a C1 to C3 alkyl-R⁵ group, wherein R⁵ is a hydrogen atom or a hydroxyl group;
n is zero or 1;
m, p are each zero.

A preferred embodiment (5) concretizing any one of embodiments (1) to (4) relates to said the compound of formula (I), which is selected from the group consisting of levomepromazine, trifluoperazine, thiethylperazine, perphenazine, chlorpromazine, thioridazine, diethazine, and mixtures of two or more thereof.

A preferred embodiment (6) concretizing any one of embodiments (1) to (5) relates to said compound of formula (I) for use according to any one of embodiment (1) to (5), wherein R³ and R⁴ form a six-membered ring comprising one further nitrogen atom, wherein the further nitrogen atom has a methyl group or a —CH₂—CH₂—OH group.

A preferred embodiment (7) concretizing any one of embodiments (1) to (6) relates to said compound of formula (I) for use according to any one of embodiments (1) to (6), wherein the compound has formula (Ia):

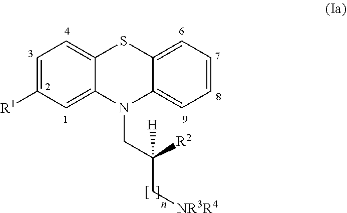

wherein
R¹ is selected from the group consisting of methoxy group, trifluoromethyl group and thioethyl group;
R² is a hydrogen atom or a methyl group (preferably having R configuration);
R³, R⁴ are both a methyl group or form together a six-membered alkyl ring, which comprises one further nitrogen atom, wherein the one further nitrogen is substituted with a methyl group,
n is zero or 1.

A preferred embodiment (8) concretizing any one of embodiments (1) to (7) relates to said compound of formula (Ia) for use according to embodiments (1) to (7), wherein R³, R⁴ are both a methyl group or form together a piperazine ring wherein the further nitrogen atom is substituted with a methyl group (4-methylpiperazin-1-yl group).

A preferred embodiment (9) concretizing any one of embodiments (1) to (8) relates to said compound of formula (I) or of formula (Ia) for use according to embodiments (1) to (8), wherein the compound of formula (I) or of formula (Ia) is used as pharmaceutically acceptable salt of a compound of formula (I) or of a compound of formula (Ia), wherein the pharmaceutically acceptable salt of a compound of formula (I) or of a compound of formula (Ia) is administered so that the part of the cation corresponding to a compound of formula (I) or of a compound of formula (Ia) amounts to ≤20 mg, preferably in the range of from 2 to 20 mg, preferably per day of administration.

A preferred embodiment (10) concretizing any one of embodiments (1) to (9) relates to said compound of formula (I) or formula (Ia) for use according to any one of embodiments (1) to (9), wherein a pharmaceutically acceptable salt of formula (I) or formula (Ia) comprises one or more anion(s) selected from the group consisting of sulfate, pyrosulfate, bisulfate, sulfite, bisulfite, phosphate, monohydrogen phosphate, dihydrogen phosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, malate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenyl acetate, phenyl propionate, phenyl butyrate, citrate, lactate, β-hydroxybutyrate, glycolate, tartrate, methanesulfonate, propanesulfonate, naphthalene-1-sulfonate, naphthalene-2-sulfonate, almondate, adipate, alginate, mucate, aspartate, benzenesulfonate, butyrate, camphorate, camphorsulfonate, cyclopentane propionate, digluconate, dodecyl sulfate, ethane sulfonate, glucoheptanoate, glycerophosphate, hemisulfate, hexanoate, 2-hydroxy ethane sulfonate, 2-napthalene sulfonate, nicotinate, nitrate, palmitate, pectinate, persulfate, picrate, pivalate, salicylate, thiocyanate, tosylate, arginate, and undecanoate.

A preferred embodiment (11) concretizing any one of embodiments (1) to (10) relates to said compound of formula (I) or formula (Ia) for use according to any one of embodiments (1) to (10), wherein a pharmaceutically acceptable salt of formula (I) or formula (Ia) comprises one or more anion(s) selected from the group consisting of chloride, sulfate, mesylate, besylate, tosylate, fumarate, malonate, malate, maleate, succinate, lactate, glycolate, citrate, aspartate and mandelate, preferably malate and/or maleate, more preferably maleate.

A preferred embodiment (12) concretizing any one of embodiments (1) to (11) relates to said compound of formula (I) or formula (Ia) for use according to any one of embodiments (1) to (11), wherein the compound of formula (I) or formula (Ia) or pharmaceutically acceptable salt of formula (I) or formula (Ia) is selected from the group consisting of levomepromazine, trifluoperazine and thiethylperazine and pharmaceutically acceptable salts of these compounds and is preferably levomepromazine or thiethylperazine or a pharmaceutically acceptable salt of levomepromazine or a pharmaceutically acceptable salt of thiethylperazine, and is more preferably thiethylperazine or a pharmaceutically acceptable salt of thiethylperazine.

A preferred embodiment (13) concretizing any one of embodiments (1) to (12) relates to said compound of formula (I) or formula (Ia) or pharmaceutically acceptable salt of formula (I) or formula (Ia) for use according to any one of embodiments (1) to (12), wherein the compound of formula (I) or formula (Ia) is used alone or as a mixture of two or more compounds of the formula (I) or formula (Ia) or as a pharmaceutically acceptable salt of a compound of the formula (I) or formula (Ia) or as a mixture of two or more pharmaceutically acceptable salts of compounds of the formula (I) or formula (Ia).

A preferred embodiment (14) concretizing any one of embodiments (1) to (13) relates to said compound of formula (I) or formula (Ia) for use according to any one of embodiments (1) to (13), wherein the compound of formula (I) or formula (Ia) is administered in a dosage in the range of from 5 to 15 mg, preferably in the range of from 6 to 13.5 mg, per day of administration.

A preferred embodiment (15) concretizing any one of embodiments (1) to (14) relates to said compound of formula (I) or formula (Ia) for use according to embodiment (1) or (14), wherein the compound of formula (I) or formula (Ia) is administered in a dosage in the range of from 6 to 7 mg per day of administration or in the range of from 12.5 to 13.5 mg per day of administration.

A preferred embodiment (16) concretizing any one of embodiments (1) to (15) relates to said compound of formula (I) or formula (Ia) for use according to any one of embodiments (1) to (15), wherein the compound of formula (I) or formula (Ia) is used as pharmaceutically acceptable salt of formula (I) or formula (Ia), preferably as thiethylperazine maleate, which is preferably administered in a dosage≤31.62 mg, more preferably in the range of from 3.16 to 31.62 mg, more preferably in the range of from 7.9 to 23.72 mg, more preferably in the range of from 9.48 to 21.34 mg, of thiethylperazine maleate per day of administration.

A preferred embodiment (17) concretizing embodiment (16) relates to said compound of formula (I) or formula (Ia) for use according to embodiment (16), wherein the compound of formula (I) or formula (Ia) is used as pharmaceutically acceptable salt of formula (I) or formula (Ia), preferably as thiethylperazine maleate, which is preferably administered in a dosage in the range of from 9.47 to 11.07 mg thiethylperazine maleate per day of administration or in the range of from 19.77 to 21.34 mg thiethylperazine maleate per day of administration.

A preferred embodiment (18) concretizing any one of embodiments (1) to (17) relates to said compound of formula (I) or formula (Ia) for use according to any one of embodiments (1) to (17), wherein the compound of formula (I) or formula (Ia) or pharmaceutically acceptable salt of formula (I) or formula (Ia) is administered as a single dose once per day of administration or twice per day of administration.

A preferred embodiment (19) concretizing any one of embodiments (1) to (18) relates to said compound of formula (I) or formula (Ia) for use according to any one of embodiments (1) to (18), wherein the compound of formula (I) or formula (Ia) or pharmaceutically acceptable salt of formula (I) or formula (Ia) is administered orally or rectally.

An embodiment (20) of the present invention relates to a pharmaceutical composition comprising the compound of formula (I) or formula (Ia) as defined in any one of embodiments (1) to (19) in an amount≤20 mg, preferably in the range of from 2 to 20 mg, more preferably in the range of from 5 to 15 mg, more preferably in the range of from 6 to 13.5 mg.

A preferred embodiment (21) concretizing embodiment (20) relates to said pharmaceutical composition comprising the compound of formula (I) or formula (Ia) according to embodiment (20), wherein the compound of formula (I) or formula (Ia) is used as pharmaceutically acceptable salt of formula (I) or formula (Ia), wherein the pharmaceutically acceptable salt of a compound of formula (I) or of a compound of formula (Ia) is used so that the part of the cation corresponding to a compound of formula (I) or of a compound of formula (Ia) amounts to ≤20 mg, preferably in the range of from 2 to 20 mg, preferably as thiethylperazine maleate, wherein the pharmaceutical composition comprises thiethylperazine maleate in an amount≤31.62 mg, preferably in the range of from 3.16 to 31.62 mg, more preferably in the range of from 7.9 to 23.72 mg, more preferably in the range of from 9.48 to 21.34 mg.

A preferred embodiment (22) concretizing embodiment (20) or (21) relates to said pharmaceutical composition according to embodiment (20) or (21), wherein the pharmaceutical composition is a unit dosage preparation. A preferred embodiment (23) concretizing any one of embodiments (20) to (22) relates to said pharmaceutical composition according to any one of embodiments (20) to (22) comprising the compound of formula (I) or formula (Ia) or pharmaceutically acceptable salt of formula (I) or formula (Ia) in the form of a tablet or a suppository.

A preferred embodiment (24) concretizing embodiment (23) relates to said pharmaceutical composition according to embodiment (23), wherein a tablet further comprises one or more auxiliary agents, preferably selected from the group consisting of gum arabic, gelatin, talc, stearic acid, corn starch, lactose, sucrose, β-carotene and peanut oil.

A preferred embodiment (25) concretizing embodiment (23) relates to said pharmaceutical composition according to embodiment (23), wherein a suppository further comprises one or more auxiliary agents, preferably selected from the group consisting of lactose, preferably lactose monohydrate and solid semisynthetic glycerides.

A preferred embodiment (26) concretizing any one of embodiments (20) to (22) relates to said pharmaceutical composition according to any one of embodiments (20) to (22) comprising the compound of formula (I) or formula (Ia) or pharmaceutically acceptable salt of formula (I) or formula (Ia) in the form of a solution, preferably a solution suitable for injection.

A preferred embodiment (27) concretizing embodiment (26) relates to said pharmaceutical composition according to embodiment (26), wherein a solution suitable for injection further comprises water and one or more components selected from the group of sodium metabisulfite, ascorbic acid, and sorbitol.

A preferred embodiment (28) concretizing embodiment (26) or (27) relates to said pharmaceutical composition according to embodiment (26) or (27), wherein a solution suitable for injection has a pH value in <5, preferably <4, more preferably <3, wherein the pH value is preferably determined by a pH sensitive glass electrode according to DIN 19263:2007-05.

A preferred embodiment (29) concretizing embodiment (28) relates to said pharmaceutical composition according to embodiment (28) comprising the compound of formula (I) or formula (Ia) or pharmaceutically acceptable salt of formula (I) or formula (Ia) in the form of an aerosol, preferably an aerosol suitable for spray application.

The present invention is further illustrated by the following reference examples, comparative examples, and examples.

### EXAMPLES

The following examples shall merely illustrate the invention. Whatsoever, they shall not be construed as limiting the scope of the invention.

“Abeta1-40” stands for human amyloid beta (1-40)
protein fragment (CAS no. 131438-79-4, peptide
sequence
DAEFRHDSGYEVHHQKLVFFAEDVGSNKGAIIGLMVGGVV.
“Abeta1-42” stands for human amyloid beta (1-42)
protein fragment (CAS no. 107761-42-2),
peptide sequence
DAEFRHDSGYEVHHQKLVFFAEDVGSNKGAIIGLMVGGVVIA.

### I Methods

### I.1 Blood Sampling

Samples of 10 ml venous blood were collected in standardized test tubes containing ethylene diamine tetraacetic acid (EDTA) and gently shaken 1-2 times. As soon as possible (within max. 30 min.) samples were centrifuged at 1600 g, room temperature for 10 min. Plasma was aliquoted into 6 portions of 0.5 ml. All aliquots were frozen into a deep freezer (−80° C.) within 10 min after the centrifugation and stored until use.

### I.2 Plasma Abeta Determination

Abeta measurements were performed using the bead-based INNO-BIA plasma Abeta forms immunoassay (Innogenetics/Fujirebio Europe, Gent, Belgium) according to the manufacturer's protocol. Plasma samples and all other kit reagents were prepared according to the manufacturer's protocol. In brief, plasma samples were thawed at room temperature, centrifuged at 4000 g for 10 min to remove any possible debris, vortexed and then diluted 3-fold using Plasma Diluent. After transferring into the filter plate diluted samples, controls and standards were washed and incubated shaking overnight at 4° C. after the addition of the first conjugate. On the next day, all wells were washed 3 times and incubated with detection conjugate for 1 h at room temperature. After another 3 times washing, reading solution was added to all wells and the measurements were performed on a Luminex IS 100 (Luminex Corporation, Austin, TX, USA) platform after 2 minutes of shaking. Luminex instrument settings used were: gate—7500 to 15000, sample volume—50 μL and counts per region—100 beads. Standard curve calculation was performed using Stata 14.2 (StataCorp, College Station, TX, USA) employing a four-parametric logistic fit (nl log4 command).

All standards, controls, and the patients' samples were analyzed in duplicates; all samples from a given individual were assayed in one analytical run. The duplicate-averaged concentrations (in pg/ml) of the two biomarkers (Abeta1-40 and Abeta1-42) were calculated. In all eight standard-curves fitting models (4 runs×2 biomarkers), adjusted R-squared statistics were higher than 0.995, which speaks for excellent curve fitting quality.

### II Experiments and Results

Herein below, thiethylperazine was used as thiethylperazine maleate, wherein the weight-based amounts [in mg] are indicated with respect to thiethylperazine; the weight-based amounts of thiethylperazine maleate [in mg] are indicated in parenthesis. Based on pre-clinical results (Wesołowska, Molnar et al. 2009, Bateman 2010, Mawuenyega, Sigurdson et al. 2010, Krohn, Lange et al. 2011, Hofrichter, Krohn et al. 2013, Roberts, Elbert et al. 2014, Krohn, Bracke et al. 2015, Jepsen, De Both et al.), 15 human subjects were treated with different doses of thiethylperazine. On day 1 (t=0 h), blood samples were taken from all subjects. Directly thereafter, the subjects received a first dose of 6.5 mg thiethylperazine (10.28 mg thiethylperazine maleate) on day 1, wherein four hours after administration blood samples were taken. Directly after blood sampling, a second dose of 6.5 mg thiethylperazine (10.28 mg thiethylperazine maleate) was administered and four hours later, blood samples were taken for the second time. After the second blood sampling, two further doses of 6.5 mg thiethylperazine (10.28 mg thiethylperazine maleate) were administered, each after four hours, with a nightly break between the fourth intake on one day and the first intake after 12 hours on the next morning, so that overall, 26 mg thiethylperazine (41.12 mg thiethylperazine maleate) were administered per day over three consecutive days. The steady state was reached between the end of day two (48 h, 4 times half-life of thiethylperazine) and the beginning of day three (52h, 5 times half-life of thiethylperazine). On day four, when the steady state was given, four hours after administration of the second dose, blood samples were taken in order to analyze the steady state thiethylperazine concentration etc. The thiethylperazine was always given orally, in the form of 1 tablet of 6.5 mg thiethylperazine (10.28 mg thiethylperazine maleate).

Plasma Abeta was considered an indicator of brain Abeta content and Abeta export with higher plasma Abeta concentrations indicating higher Abeta clearance from the brain (Ferrero, Williams et al. 2016, Sevigny, Chiao et al. 2016). Plasma Abeta was determined after the subjects were treated with doses of 6.5 (10.28 mg thiethylperazine maleate) and 13 mg (20.56 mg thiethylperazine maleate) thiethylperazine as well as after 3 days of taking 26 mg/d thiethylperazine (comparative) (41.12 mg thiethylperazine maleate).

After taking the initial dose of 6.5 mg thiethylperazine (10.28 mg thiethylperazine maleate) on day 1, an increase of plasma Abeta1-40 was found in 53% of the subjects (8 of 15). After the second administration on day 1, i.e. after a dose of 13 mg thiethylperazine (20.56 mg thiethylperazine maleate), plasma Abeta1-40 was increased in 87% of the subjects (13 of 15). Both doses led to a similar average 7.8% increase in plasma Abeta1-40.

26 mg thiethylperazine (41.12 mg thiethylperazine maleate) per day over three consecutive days only led in the steady state to an increase of plasma Abeta1-40 in 40% of study subjects (6 of 15) with an average change of 6.5%.

That is, compared to only 40% of study subjects (6 of 15) showing an increase of plasma Abeta1-40 significantly more subjects, i.e. in the range of 30 to 117% more subjects, were responsive to a lower dose of thiethylperazine in the range of from 2-20 mg, here especially of 6.5 mg (10.28 mg thiethylperazine maleate) and 13 mg thiethylperazine (20.56 mg thiethylperazine maleate) respectively.

Furthermore, also the average increase in plasma Abeta1-40 was significantly higher, especially ≥20% higher compared to subjects, which had been treated with the higher amount of 26 mg thiethylperazine (41.12 mg thiethylperazine maleate).

The mean plasma Abeta1-40 concentration difference between responders and non-responders was 33.04 pg/ml, 28.07 pg/ml and 16.53 pg/ml respectively for 6.5, 13 and 26 mg thiethylperazine (10.28 mg, 20.56 mg, 41.12 mg thiethylperazine maleate). That is, also the mean plasma Abeta1-40 concentration difference between responders and non-responders was substantially increased by ≥10 pg/ml (or ≥60%) in subjects who had been treated with a lower dose of thiethylperazine in the range of from 2-20 mg, here especially of 6.5 mg (10.28 mg thiethylperazine maleate) and 13 mg thiethylperazine (20.56 mg thiethylperazine maleate) respectively, compared to subjects, which had been treated with the higher amount of 26 mg thiethylperazine (41.12 mg thiethylperazine maleate) in steady state mode. Both, Abeta1-40 difference and thiethylperazine dose correlated highly with thiethylperazine plasma levels (Abeta1-40 R²=−0.999, dose R²=0.998).

Hence, it was shown that surprisingly, the efficacy and response to a thiethylperazine treatment in humans improved towards lower, not higher doses of thiethylperazine.

The results of the determination of the Abeta plasma concentrations after treatment are shown in FIG. 1.

Inter-assay precision was estimated on the ground of four repetitions of the two quality control (QC) samples (A+B), and resulted in the coefficients of variation (CV) presented below in Table 1:

TABLE 1
Coefficients of variation

| Assay | Sample | Abeta1-40 | Abeta1-42 |
|---|---|---|---|
| 1 | A | 44.05 | 152.22 |
| 1 | B | 117.51 | 22.40 |
| 2 | A | 40.43 | 144.22 |
| 2 | B | 101.85 | 21.10 |
| 3 | A | 41.51 | 151.82 |
| 3 | B | 114.33 | 22.31 |
| 4 | A | 43.61 | 144.46 |
| 4 | B | 115.93 | 21.37 |

Inter-assay precision was estimated on the ground of four repetitions of the two QC samples, and resulted in the coefficients of variation (CV) for A or B presented below in Tables 2 and 3:

TABLE 2
Coefficients of variation for A

| Control A | Abeta1-40 | Abeta1-42 |
|---|---|---|
| Mean (pg/ml) | 42.4 | 148.2 |
| CV | 4.1% | 3.0% |

TABLE 3
Coefficients of variation for B

| Control B | Abeta1-40 | Abeta1-42 |
|---|---|---|
| Mean (pg/ml) | 112.4 | 21.8 |
| CV | 6.4% | 3.0% |

Intra-assay precision was estimated as medians and inter-quartile ranges (p25-p75) of the differences between the duplicate measurements divided by their averages (“Range-To-Average”) of all samples, and is presented below in Table 4:

TABLE 4
Intra-assay precision

| | Abeta1-40 | Abeta1-42 |
|---|---|---|
| p25 | 1.6% | 0.8% |
| Median | 3.1% | 1.8% |
| p75 | 5.8% | 3.0% |

### SHORT DESCRIPTION OF THE FIGURE

FIG. 1 shows individual plasma Abeta concentrations in treatment responders before (pre) and after (post) different doses of thiethylperazine administration. Asterisks indicate levels of significance of mean-pre vs. mean-post thiethylperazine administration (*p≤0.05, **p≤0.01, ***p≤0.001).

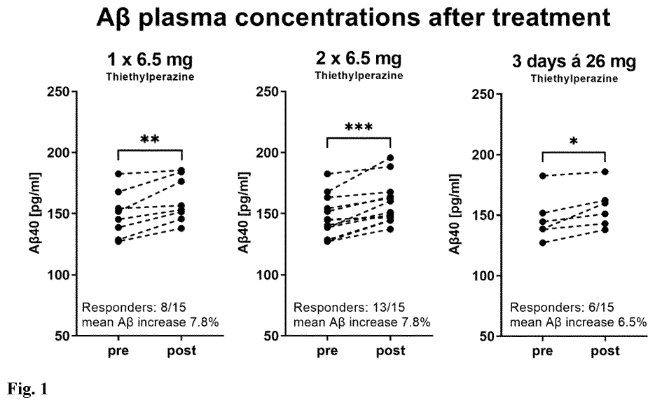

### CITED LITERATURE

Bateman, R. J. (2010). “O2-03-05: Amyloid-beta production and clearance rates in Alzheimer's disease.” Alzheimer's & Dementia 6(4): S101-S101.
Ferrero, J., L. Williams, H. Stella, K. Leitermann, A. Mikulskis, J. O'Gorman and J. Sevigny (2016). “First-in-human, double-blind, placebo-controlled, single-dose escalation study of aducanumab (BIIB037) in mild-to-moderate Alzheimer's disease.” Alzheimer's & Dementia: Translational Research & Clinical Interventions 2(3): 169-176.
Hofrichter, J., M. Krohn, T. Schumacher, C. Lange, B. Feistel, B. Walbroel, H. J. Heinze, S. Crockett, T. F. Sharbel and J. Pahnke (2013). “Reduced Alzheimer's disease pathology by St. John's Wort treatment is independent of hyperforin and facilitated by ABCC1 and microglia activation in mice.” Curr. Alzheimer Res. 10(10): 1057-1069.
Jepsen, W. M., M. De Both, A. L. Siniard, K. Ramsey, I. S. Piras, M. Naymik, A. Henderson and M. J. Huentelman (2020). “Adenosine triphosphate Binding Cassette subfamily C member 1 (ABCC1) overexpression reduces APP processing and increases alpha- versus beta-secretase activity, in vitro.” Biol Open: bio.054627.
Krohn, M., A. Bracke, Y. Avchalumov, T. Schumacher, J. Hofrichter, K. Paarmann, C. Fröhlich, C. Lange, T. Brüning, O. von Bohlen Und Halbach and J. Pahnke (2015). “Accumulation of murine amyloid-beta mimics early Alzheimer's disease.” Brain 138(Pt 8): 2370-2382.
Krohn, M., C. Lange, J. Hofrichter, K. Scheffler, J. Stenzel, J. Steffen, T. Schumacher, T. Bruning, A. S. Plath, F. Alfen, A. Schmidt, F. Winter, K. Rateitschak, A. Wree, J. Gsponer, L. C. Walker and J. Pahnke (2011). “Cerebral amyloid-beta proteostasis is regulated by the membrane transport protein ABCC1 in mice.” J Clin Invest 121(10): 3924-3931.
Mawuenyega, K. G., W. Sigurdson, V. Ovod, L. Munsell, T. Kasten, J. C. Morris, K. E. Yarasheski and R. J. Bateman (2010). “Decreased clearance of CNS beta-amyloid in Alzheimer's disease.” Science 330(6012): 1774.
Roberts, K. F., D. L. Elbert, T. P. Kasten, B. W. Patterson, W. C. Sigurdson, R. E. Connors, V. Ovod, L. Y. Munsell, K. G. Mawuenyega, M. M. Miller-Thomas, C. J. Moran, D. T. Cross, 3rd, C. P. Derdeyn and R. J. Bateman (2014). “Amyloid-beta efflux from the central nervous system into the plasma.” Ann Neurol 76(6): 837-844.
Sevigny, J., P. Chiao, T. Bussière, P. H. Weinreb, L. Williams, M. Maier, R. Dunstan, S. Salloway, T. Chen, Y. Ling, J. O′Gorman, F. Qian, M. Arastu, M. Li, S. Chollate, M. S. Brennan, O. Quintero-Monzon, R. H. Scannevin, H. M. Arnold, T. Engber, K. Rhodes, J. Ferrero, Y. Hang, A. Mikulskis, J. Grimm, C. Hock, R. M. Nitsch and A. Sandrock (2016). “The antibody aducanumab reduces Abeta plaques in Alzheimer's disease.” Nature 537(7618): 50-56.
Wesołowska, O., J. Molnar, I. Ocsovszki and K. Michalak (2009). “Differential effect of phenothiazines on MRP1 and P-glycoprotein activity.” In Vivo 23(6): 943-947.
Lieberman, R., Nelson R. (1993). “Dose-Response and Concentration-Response Relationships: Clinical and Regulatory Perspectives” Therapeutic Drug Monitoring 15:498-502

1-10. (canceled)

11. A method for treating and/or preventing Alzheimer's dementia in a subject, wherein the method comprises:
administering a compound of formula (I); 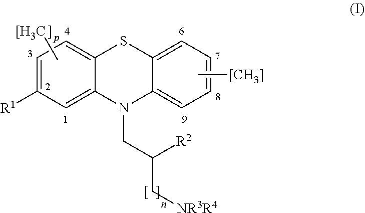
wherein:
R¹ is selected from the group consisting of hydrogen atom, halogen atom, methoxy group, perhalogenated alkyl group and —X-C1 to C2 alkyl group, wherein X is either a sulphur atom or an oxygen atom;
R² is a hydrogen atom or a methyl group;
R³, R⁴ are independently of each other either a hydrogen atom or C1 to C4 alkyl group or form together a six membered alkyl ring which comprises at least one further nitrogen atom, wherein the at least one further nitrogen is substituted with a C1 to C3 alkyl-R⁵ group, wherein R⁵ is a hydrogen atom or a hydroxyl group;
n is zero or 1;
m is zero or an integer selected from the range of from 1 to 4;
p is zero or an integer selected from the range of from 1 to 3;
wherein the compound of formula (I) is administered in a dosage≤20 mg per day of administration.

12. The method of claim 11, wherein the compound of formula (I) is administered in a dosage in the range of from 2 to 20 mg per day of administration.

13. The method of claim 11, wherein the compound of formula (I) is a compound that has formula (Ia): 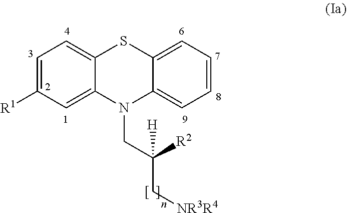
wherein
R¹ is selected from the group consisting of methoxy group, trifluoromethyl group and thioethyl group;
R² is a hydrogen atom or a methyl group;
R³, R⁴ are both a methyl group or form together a six membered alkyl ring, which comprises one further nitrogen atom, wherein the one further nitrogen is substituted with a methyl group,
n is zero or 1.

14. The method of claim 11, wherein the compound of formula (I) is administered in a dosage in the range of from 5 to 15 mg per day of administration.

15. The method of claim 11, wherein the compound of formula (I) is a pharmaceutically acceptable salt of formula (I), wherein the pharmaceutically acceptable salt comprises one or more anion(s) selected from the group consisting of chloride, sulfate, mesylate, besylate, tosylate, fumarate, malonate, malate, maleate, succinate, lactate, glycolate, citrate, aspartate and mandelate.

16. The method of claim 11, wherein the compound of formula (I) is selected from the group consisting of levomepromazine, trifluoperazine, thiethylperazine, and pharmaceutically acceptable salts of these compounds.

17. The method of claim 11, wherein the compound of formula (I) is thiethylperazine maleate, and wherein the thiethylperazine maleate is administered in a dosage≤31.62 mg.

18. The method of claim 11, wherein the compound of formula (I) is administered orally or rectally.

19. The method of claim 11, wherein the compound of formula (I) is administered as a pharmaceutical composition comprising the compound of formula (I) and a pharmaceutically acceptable excipient, wherein the preparation contains in the range of 2 to 20 mg of the compound of formula (I),.

20. The method of claim 19, wherein the pharmaceutical composition is in the form of a solution suitable for injection.

21. The method of claim 15, wherein formula (I) of the pharmaceutically acceptable salt of formula (I) is selected from the group consisting of levomepromazine, trifluoperazine, thiethylperazine.

22. The method of claim 15, wherein the pharmaceutically acceptable salt of formula (I) is administered orally or rectally.

23. The method of claim 15, wherein the pharmaceutically acceptable salt of formula (I) is administered as a pharmaceutical composition comprising the pharmaceutically acceptable salt of formula (I) and a pharmaceutically acceptable excipient, wherein the preparation contains in the range of 2 to 20 mg of the compound of formula (I).

24. The method of claim 11, wherein the compound of formula (I) is administered in a dosage in the range of 6 to 13.5 mg per day of administration.

25. The method of claim 11, wherein the compound of formula (I) is administered in a dosage in the range of 6 to 7 mg per day of administration.

26. The method of claim 11, wherein the compound of formula (I) is administered in a dosage in the range of 12.5 to 13.5 mg per day of administration.

27. The method of claim 11, wherein the compound of formula (I) is a pharmaceutically acceptable salt of formula (I), wherein the pharmaceutically acceptable salt comprises one or more anion(s) selected from malate and/or maleate.

28. The method of claim 11, wherein the compound of formula (I) is a pharmaceutically acceptable salt of formula (I), wherein the pharmaceutically acceptable salt comprises the anion maleate.

29. The method of claim 11, wherein the compound of formula (I) is selected from the group consisting of levomepromazine, and thiethylperazine.

30. The method of claim 11, wherein treating Alzheimer's dementia comprises increasing the subject's plasma Abeta1-40 concentration.
